(19) United States
(12) Patent Application Publication
(10) Pub. No.: US 2024/0164356 A1
(43) Pub. Date: May 23, 2024

(54) PROCESS FOR BREEDING ADULT INSECTS

(71) Applicant: NASEKOMO B.V., Amsterdam (NL)

(72) Inventor: Marc Louis Raymond BOLARD, Sofia (BG)

(21) Appl. No.: 18/548,867

(22) PCT Filed: Mar. 25, 2022
(86) PCT No.: PCT/EP2022/057870

§ 371 Date: Sep. 1, 2023

(30) Foreign Application Priority Data
Mar. 26, 2021 (EP) 21165414.0

(51) Int. Cl. A01K 67/033

(52) U.S. Cl. CPC A01K 67/0339

(57) ABSTRACT

An automated process for breeding insects at their imaginal stage in at least one mating cage, comprises the steps of: providing at least one emergence chamber comprising a population of newly emerged insects; automatically successively: connecting at least one emergence chamber with at least one mating cage, filling the at least one mating cage with at least part of the population of newly emerged insects during a first time interval T1, and disconnecting the at least one mating cage and the at least one emergence chamber; providing conditions within the at least one mating cage suitable for promoting ovipositing during a second time interval T2; stopping ovipositing during a third time interval T3.

### FIELD OF THE INVENTION

The present invention relates to the field of insect breeding, in particular the production of Dipteran insects.

More specifically, the invention relates to an automated cycle for controlling the reproduction of adult insects, between the pupation stage and the death of these insects, as well as the collection of their eggs.

### TECHNOLOGICAL BACKGROUND

Insects, in particular certain species, can constitute a source of products or of raw materials, in particular for animal feed or human foodstuffs. They are particularly well-suited to converting organic materials, such as commercial or residential waste products, or low quality biomass into foodstuff of high nutritional quality, with high protein content.

The optimization of the insect breeding is in consequence of paramount importance to ensure the sustainable food needs of our planet.

In the field of insect rearing, it is known to create devices or even farms in which the different stages of insect life are managed at different places, at each of which the environmental conditions are optimized for a given stage.

WO2019/053456 A1 is an example of a modular system for fly breeding, in which the life of insects is subdivided into discrete stages forming a cycle. A batch of insects, or a combination of batches of insects, goes through these different stages, for each of which the humidity, temperature and lighting conditions are controlled. In particular, the stages are distinguished: “egg growth chamber”, “larval chamber”, “pupation chamber”, “emergence box” and “reproduction chamber”.

However, this device does not disclose any details of what occurs in the “reproduction chamber”.

WO2016/011541 A1 discloses a mating chamber for insects, in particular Hermetica Illucens, comprising several successive automated steps, all identical to each other. A given pupation chamber enters from one side of the chamber, advances one stage per day so that it replaces the pupation chamber which precedes it and it is replaced by the pupation chamber which follows.

After n days spent in the mating chamber, the pupation chamber exits the mating chamber. This device is only operable in a FIFO mode, and it does neither give any detail of the conditions or the operations occurring in the mating chamber.

U.S. Pat. No. 10,405,528B2 discloses an automated farm for insect breeding, comprising a plurality of interchangeable stations, among: feeding the insects, providing them with water, renewing the substrate, identifying insects having a disease, killing surplus, diseased and/or contaminated insects, etc. However, this document doesn't disclose a cycle including one or more of these steps, this cycle being designed in order to optimize the mating step of the insects.

Hence, this invention aims at providing a fully automated mating cycle in order to optimize the mating step and the collect of the eggs and permitting specific operations or specific tuning at each given step of the cycle if needed.

### SUMMARY OF THE INVENTION

The invention relates to an automated process for breeding insects at their imaginal stage in at least one mating cage. This process comprises:

1) providing at least one emergence chamber comprising a population of newly emerged insects;
a) automatically successively:
   connecting at least one emergence chamber with at least one mating cage,
   filling the at least one mating cage with at least part of the population of newly emerged insects during a first time interval T1, and
   disconnecting the at least one mating cage and the at least one emergence chamber;
b) providing conditions within at least one of the at least one mating cage suitable for promoting ovipositing during a second time interval T2;
c) stopping ovipositing during a third time interval T3.

Automatically connecting the mating cage with at least one emergence chamber, filling the mating cage and disconnecting the mating cage and the emergence chamber makes it possible to automatically control the population filling the mating cage and to implement the breeding process on an industrial scale.

Stopping ovipositing makes it possible to optimize the oviposition yield, since most of the ovipositing occur in the early days of female adult life. The oviposition yield grows indeed much faster in the first days of life of the adults than at the end of their life.

This stopping step also makes it possible to build a periodic cycle that can be easily managed in an automated manner.

In a particular embodiment, the process further comprises performing a calibration before 1).

The calibration comprises:
i) filling at least one mating cage with a population of newly emerged insects;
ii) providing conditions suitable for promoting mating and/or oocytes maturation and/or ovipositing within said at least one mating cage during each day of at least three days;
ii) providing at least one oviposition device at least at the beginning of each of the at least three days for each of the at least one mating cage;
iv) collecting at least one oviposition device at least at the end of each of the at least three days for each of the at least one mating cage;
v) measuring the weights of the eggs collected in each of the oviposition device (2);
vi) determining a duration between the end of a) and the beginning of c) based on the weights measured at v);
a), b) and c) being subsequently performed in such a way as to respect said duration.

Thanks to the calibration, the process can be optimized at least in terms of egg yield. Even if the type of insects is known, the daily egg yield can be different from one batch of insects to another batch, from one rearing location to another rearing location, and/or from one series of conditions in the mating cages to another one. As a consequence, an in-place calibration for a given first generation of insects to be reared is necessary to determine the duration between the end of a) and the beginning of c) that allows the best average egg yield, in particular in a steady state when the process is cyclically iterated.

In a particular embodiment, during b), one or more oviposition devices are provided and/or collected without opening the at least mating cage.

This prevents the loss of adults and contributes to optimize the egg yield.

In a particular embodiment, the process also comprises after c):
d) cleaning and emptying one mating cage during a fourth time interval T4 so that after d), the mating cage is suitable for a new a), and optionally:
e) storing the mating cage during a fifth time interval T5.

This makes it possible to reuse the same mating cage periodically in good sanitary conditions.

In a particular embodiment, the process further comprises f):
providing conditions within at least one mating cage suitable for promoting mating during and/or oocytes maturation and/or ovipositing during a sixth time interval T6 between b) and c).

The conditions of f) can differ from the conditions of b).

This makes it possible to fine-tune the conditions either for mating, or for oocytes maturation or for ovipositing, or for a combination of two of these stages, and as a consequence, improve the egg-yield.

In a particular embodiment, the duration between the beginning of a) and the beginning of c) is shorter than the life expectancy of the adult insects after emergence.

This allows restarting a new process with the same mating cage with higher daily egg yields than if one had awaited the death of the population filling the mating cage. As a consequence, if the process is reiterated at least two times, the average egg yield with the same mating cage is higher than in the case the duration between the beginning of a) and the beginning of c) had been longer than the life expectancy of the adult insects after emergence.

In a particular embodiment, c) starts once at least one of the following threshold is met: the percentage of dead adults meets a first threshold, the percentage of females having oviposited meets a second threshold, the egg yield meets a third threshold.

These two embodiments make it possible to optimize the egg-yield by not waiting for the death of the adults.

In a particular embodiment, at least two of the time intervals of the process are equal.

This makes it possible to synchronize several steps of the process and to launch several staggered cycles in case the process is cyclically iterated.

In a particular embodiment, at least two of 1), a), b), c) and optionally (or in other words if applying), d), e), f), take place at at least two different places, and the mating cage is moved from one of these places to another if these places of the at least two different places between two 1), a), b), c) and optionally d), e), f).

In this case, several mating cages can be at several steps of the process at the same time.

In a particular embodiment, at least one condition of at least one of b) or optionally f) is chosen among an ambient temperature, a relative humidity, lighting, feeding, and watering.

These conditions are known to inhibit or enhance ovipositing and/or oocyte maturation and/or mating. In consequence, adjustment of these conditions at each step helps improve the egg-yield.

In a particular embodiment, the population of newly emerged insects filling the mating cage is selected according to at least one criterion among a sex ratio, a phenotype, a genotype, a size of the insects at the larval stage at a given date, a sexual maturity, and an age.

This makes it possible to control, or even to homogenize, the quality of the eggs collected and to optimize the egg-yield.

The invention also relates to an automated system for breeding insects at their imaginal stage, comprising:
at least one mating cage,
a filling system configured to connect at least one mating cage with at least one emergence chamber comprising a population of newly emerged insects, to fill the at least one mating cage with at least part of a population of newly emerged insects comprised in at least one emergence chamber during a first time interval T1 and to disconnect the at least one mating cage and the at least one emergence chamber;
a provision system to provide conditions within the at least one mating cage suitable for promoting ovipositing during a second time interval T2;
a terminating system to stop ovipositing during a third time interval T3.

In a particular embodiment, the automated system further comprises a second provision system to provide conditions within the at least one mating cage suitable for promoting mating and/or oocytes maturation and/or ovipositing.

In a particular embodiment, the automated system further comprises a connection system configured to allow a temporary connection of at least one mating cage with at least one emergence chamber comprising, and as a consequence to allow the disconnection of the at least one emergence chamber and the at least one mating cage.

In a particular embodiment, of the automated system, the at least one mating cage:
comprises at least one door comprising an automated device for its closing and opening,
is provided with at least one removable ovipositing device (2), and
has at least two discrete positions,
   and the automated system further comprises a moving device configured for moving at least one mating cage from at least one of the at least two discrete positions to at least another of the at least two discrete positions, each discrete position being equipped to automatically perform at least one of:
a) connecting at least one mating cage with at least one emergence chamber, filling at least one mating cage with at least part of the population of newly emerged insects during a first time interval T1, and disconnecting said at least one mating cage from the at least one emergence chamber;
b) providing conditions within said at least one mating cage suitable for promoting ovipositing during a second time interval T2;
c) stopping ovipositing during a third time interval T3;
   and optionally
f) providing conditions within at least one mating cage suitable for promoting mating and/or oocytes maturation and/or ovipositing.

Thus, the automated system further comprises a device for moving the at least one mating cage from at least one of the at least two discrete positions to at least another of the at least two discrete positions, each discrete position being equipped to automatically perform at least one step of the automated process for breeding insects at their imaginal stage.

The door makes it possible to automatically connect the mating cage with an emergence chamber.

The discrete positions allow the setting of different conditions at each position, specifically adjusted for a given step, rather than changing these conditions at the same location for a mating cage remaining there.

In one embodiment of the automated system, each of the at least one mating cage is provided with an automated device for counting the population of newly emerged insects and the automated device for closing and opening the door of the respective mating cage receives a signal from the automated device for counting the population of newly emerged insects that triggers the closing and/or opening of the door of the respective mating cage.

In this case, the filling of the mating cage can be completely automated.

In particular, the automated device for counting the adult population allows the monitoring of the filling of each mating cage and the automatic triggering of the closing and/or opening of the door of each mating cage.

In a particular embodiment of the automated system, a tracking device is provided for each of the at least one mating cage.

In a particular embodiment, the automated system further comprises a controller and a tracking device is provided for each mating cage. The controller receives tracking data from at least one tracking device of a mating cage and controls the moving of the mating cage based on the tracking data.

This allows for example to automatedly control the filling of the mating cage, and the moving of a mating cage from one step to another.

In a particular embodiment of the automated system, the device for moving a mating cage is a conveyor belt or a rail or a suspended conveyor rail or an automated guided vehicle.

In a particular embodiment of the automated system, the automated device for counting the population comprises a weight sensor.

This allows an easy monitoring of the filling of the mating cage, for example when the mating cage is suspended with a hook.

In a particular embodiment of the automated system, the at least one mating cage is made of a mesh made of metal or fabric or plastic.

This allows ventilation, cleaning and oviposition on oviposition devices on the outer side of the mating cage.

In a particular embodiment of the automated system, at least one removable oviposition device is provided for each of the at least one mating cage. The removable oviposition device can be fixed on the outer side of said mating cage.

In this case, the collection of the oviposition devices can occur at any time, it is easier and there is no loss of adults during such an operation.

In a particular embodiment of the automated system, a tracking device is provided for each of the removable oviposition devices.

In a particular embodiment, the system further comprises a controller receiving data from at least one tracking device of an oviposition device and controls the fixing of the oviposition device based on the tracking data received from the tracking device of the oviposition device.

This allows an automated collection of the eggs and the monitoring of it. In particular, the egg-yield can be studied at each collection operation and in consequence, the collection can be optimized.

The invention also relates to a system comprising a system an automated system for breeding insects at their imaginal stage coupled with a system for breeding insects at the egg stage and/or at the pupation stage.

A system for breeding insects at the pupation stage produces the population for filling the mating cage according to the invention. The eggs collected in a system for breeding insects at their imaginal stage according to the invention can be used in a system for breeding insects at the egg stage.

As a consequence, the global system can be operated cyclically and automatically.

### BRIEF DESCRIPTION OF THE DRAWINGS

Embodiments of the invention will be described below, in relation to the following drawings

FIG. 1 is a view of a mating cage with which the process can be implemented.

FIG. 2 is a view of an oviposition device.

FIG. 3 shows a mating cycle according to one embodiment.

FIG. 4 shows the contribution of each day of ovipositing in the mating cage to the egg production of a mating cage without the stopping step.

On the drawings, the same reference signs denote the same or similar objects.

### DETAILED DESCRIPTION

The invention deals with the optimization of insects rearing.

In general, Dipteran insects, such as the Black Soldier Fly (Hermetica Illucens), can be bred according to the invention described below, but other insects with a flying stage could be considered.

Black Soldier Flies are particularly well adapted for farming, since they don't bite nor sting, they aren't likely to function as a vector for diseases since they aren't attracted to human environments, and they have a high rate of conversion of biomass into protein.

The life cycle of an insect comprises four stages: the egg stage, the larval stage, the nymph stage in the case of Endopterygota, the adult or imaginal stage (adult insect and imago being synonymous).

For Dipteran insects, the stage that precedes the adult stage, or imaginal stage, is called “pupal stage” rather than nymph stage. The pupal stage follows the larval stage and the transition between the pupal stage and the adult stage is called the emergence.

The invention concerns more specifically a process for managing the imaginal phase, from the emergence until the death of the imago.

According to one embodiment, the process comprises at least the following steps:
Step 1: an empty mating cage 1 is connected to at least one emergence chamber, so as to be filled with a population of newly emerged adults.
Step 2: once filled, the mating cage 1 undergoes a first lighting cycle designed to promote the insects mating and oocyte maturation; the mating cage 1 is equipped with one or more oviposition devices 2 on which the gravid females can oviposit their fertilized eggs.
Step 3: the mating cage 1 undergoes a second lighting cycle designed to promote the insects mating and the oocyte maturation; the mating cage 1 is equipped with one or more oviposition devices 2 on which the gravid females can oviposit their fertilized eggs. Some oviposition devices 2 are collected at the beginning of or during or at the end of step 3;
Step 4: the mating cage 1 is equipped with one or more oviposition devices 2 on which the gravid females can oviposit their fertilized eggs. Some oviposition devices 2 are collected at the beginning of or during or at the end of step 4. The mating cage undergoes a third lighting cycle designed to promote oviposition;
Step 4′ (optional): the mating cage 1 is equipped with one or more oviposition devices 2 on which the gravid females can oviposit their fertilized eggs. Some oviposition devices 2 are collected at the beginning of or during or at the end of step 4′. Mating cage 1 undergoes a fourth lighting cycle designed to promote oviposition.
Step 5: After step 4 or step 4′, the adults that are not already dead are killed before their natural death;
Step 6: the mating cage 1 is cleaned before entering a new cycle.

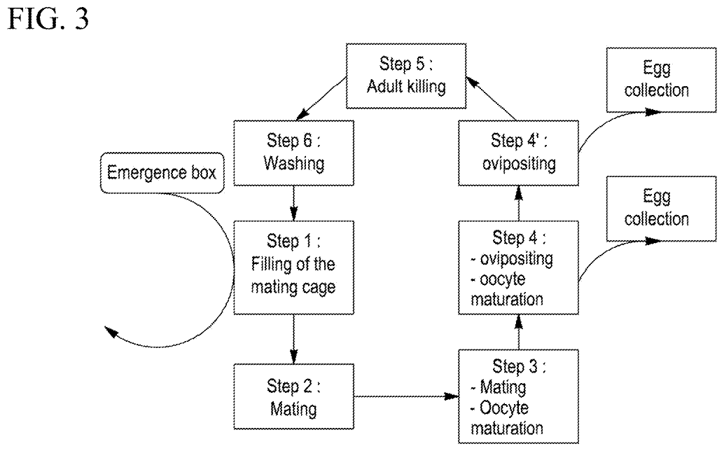

An exemplary embodiment of the process is shown on FIG. 3.

The process can be implemented on a device comprising at least one mating cage (or mating chamber) 1, in which any given adult undergoes step 1 to step 5.

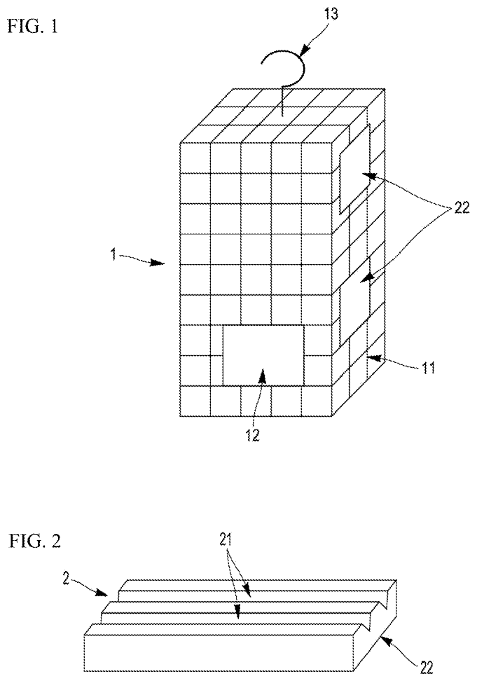

In an exemplary but non limitative embodiment, in relation to FIG. 1, a mating cage (or mating chamber) 1 is a mesh parallelepipedic box. The dimensions of the box can be adapted to the insect being reared.

For example, since adult black fly soldier fly less than 10% of the time, the height of the box can be larger than the width and length, in order to have both a high surface-to-volume ratio, since mating takes place on the surface of the box, and a sufficient height for the mating dance (or Jekking behavior), which is mainly vertical and precedes mating.

In one embodiment, a mating chamber 1 has a length and a width of the same order of magnitude and a height around 1 to 10 times the length or the width.

The mesh 11 can be made of metal or plastic or fabric or fiberglass for example. The size of the mesh is chosen so as to prevent adults from escaping and to permit oviposition on a device outside the box. It will be understood that the scale was not respected for the mesh size on FIG. 1, in order to make FIG. 1 more readable.

Optionally, part or all of the side walls and/or of the bottom of the mating cage 1 are opaque and made of the same or a different material than the other parts of the walls. This disposition helps to attract the flies to a brighter part of the cage when the light inside the box is on.

In one embodiment, at least two steps of the process are operated at two different places.

In one embodiment, each step of the process is operated at a different location.

In one embodiment, the process is automated. For this purpose, a mechanism may be provided for passing a mating chamber from one location to the following.

For example, the mating chamber can be mounted on a conveyor belt or a rail or any equivalent suitable device. In another embodiment, automated guided vehicles are provided, to automatically carry the mating chamber from one location to the following.

In one embodiment, the mating chamber is provided with a hook 13 or any suitable means, so that it can be moved automatically in a suspended monorail conveyor. This disposition makes it possible to leave the floor space to be left free for other operations, such as maintenance, surveillance, or even other life stages.

This disposition makes it also possible to control the air flux in the mating cage.

In one embodiment, the air flux is not too high, so as not to inhibit mating by dispersing the pheromones outside the mating cage 1 and complicating the mating dance for insects as clumsy as the Black Soldier Fly.

In one embodiment, step 1 comprises a sub-step of connection with one emergence chamber. By emergence chamber, a skilled man will understand any container suitable for the emergence of the imago, and possibly part or all of the pupal stage. Such an emergence chamber (or equivalently box or cage) comprises a door or a hatch, the closing and opening of which is automated, and that can be connected to the mating cage.

The mating cage also comprises at least one door or hatch 12, for example a sliding door or hatch, the closing and opening of which is automated, and through which the mating cage 1 can be connected to an emergence chamber.

The door 12 can be, in an exemplary embodiment, positioned on the underside or on the side faces of the mating cage 1.

In one embodiment, if the mating cage 1 is suspended, filling of the mating cage can be performed through a hatch on the underside of the mating cage 1, with the emergence chambers being moved from one location to another at a lower floor than the mating boxes.

At the beginning of step 1, the door 12 of a mating cage and the one of an emergence chamber containing newly emerged imagoes are tightly placed face to face, so that no adult can escape from the assembly formed by the two boxes. For example, the frames of the respective doors of the mating cage and the emergence chamber can be directly clipped to each other. The doors are simultaneously or successively opened so as to allow the newly emerged adults to pass from of the emergence chamber into the mating cage 1, for example in response to a stimulus, like a light stimulus.

The size and other characteristics of the pathway between the emergence chamber and the mating cage 1 can be chosen so that only capable adults can reach the mating cage 1.

In one embodiment, only adults able to fly can reach the mating cage 1 from the emergence chamber. Adults that are still incapable to fly will remain in the emergence chamber and will either be killed when the emergence chamber needs to be emptied before entering a new emergence cycle, or be allowed to fly into another mating cage 1 to which the emergence chamber is later connected. This disposition contributes to an even selection of high quality adults (approximately same age or development stage, no disease nor defect, etc.). In other words, the adults entering the mating cage 1 are synchronous at the beginning of the mating cycle and ready for mating. This disposition enables a minimum duration of the mating cycle.

The movement of insects can be promoted by different means, for example chemical means or more intense lighting in the mating cage than in the emergence box.

The parameters chosen to promote the movement of insects toward the mating cage also allows for selection of the population according to its phenotype (size, form of the wings, etc.).

Once the wished population has entered the mating cage 1 and/or after a predefined connection time, the door 12 and the door of the emergence chamber can be automatically closed, either simultaneously or successively.

The count of the population in the mating cage can be automated.

For example, the count of the population is based on the detection that the flies cross a light beam between the emergence chamber and the mating cage 1.

In case the mating cage 1 is mounted on a suspended rail, the hook 13 can be provided with a weight sensor. The closing of the door 12 can then be controlled by the weight of the mating cage 1.

The estimated population in the cage may be estimated from the weight of the mating cage 1, the known average weight of an insect at emergence stage, and a known ratio of flying time for an insect.

The mating cage 1 can also be weighed in darkness, so that no fly is flying when the measurement is taken.

The mating cage 1 can be designed to promote mating: for a given volume, its height can be up to 2, 3, 5, 7 or 10 times its length or width so that the height is sufficient for the mating dance.

In a particular embodiment, a given mating cage 1 can be connected successively to several emergence chambers. This disposition makes it possible to mix several larval populations, in order to select or mix particular genotypes or to limit the drifts associated with the rapid life cycle of the insects, such as the selection and reproduction of an unwanted genetic heritage.

Consequently, the filling of a mating cage 1 is relatively reproducible in terms of population characteristics, which allows a better control of the quality of the products associated with the breeding, such as foodstuff, lipids or any commercially valuable byproduct.

The sex ratio can for example be controlled, insofar as the pupal stage of a male is different than that of a female. In the case of Hermetica Illucens, the pupal stage of a male is about two days shorter than that of a female. In average, if males emerge in a given emergence chamber at day d, females will emerge at day d+2. Hence, one given mating cage 1 can be connected with a first emergence box being at the stage corresponding to day d and with a second emergence box being at the stage corresponding to day d+2. More precise distribution may be controlled based on known emergence age curves for the insect.

The duration of the first and the second connection can be set so that at the end of the filling, the female-to-male ratio is around 51% to 65%. This disposition increases the yield of the mating cycle in terms of eggs/cycle.

Other techniques, based on sexual dimorphism, can be considered to control the sex ratio.

Other selection operations may be carried out at the larval stage. For example, the larvae may be sieved in order to select larvae having the same size, and therefore of the same age (or at least the same stage of development).

Other selection operations may be carried out on the basis of the genotype. For example, the eggs of the first generation can be selected on genotypic criteria.

One understands as a consequence that one mating cage 1 can be connected successively to several emergence chambers and that reciprocally, an emergence chamber can be connected successively to several mating cages 1.

Typically, the total time (T1) for the filling of the mating cage 1 ranges from ten minutes to an hour.

Once the wished population has filled the mating cage 1, the mating cage 1 can enter step 2, at the same or at a different location, where the adult insects are subjected to a first lighting cycle to promote their mating. If the second step takes place in another location, the mating cage 1 can be automatically moved from the first to the second location.

A fine tuning of the filling of the mating chamber 1 helps having synchronized adults, that is to say adults having a homogeneous sexual maturity. This arrangement allows a fine tuning of the mating cage 1 environment at each of the following steps, on the contrary to prior art process, in which some adults mate, oviposit and die continuously in the same box.

In the process according to the invention, each step can be specifically designed for a particular step of the adult life: by way of an example, the design of the steps of the process can first focus on mating, then on egg maturation and eventually on oviposition.

The first lighting cycle lasts for 24 hours (time period T2) in an exemplary embodiment.

In general, intense lighting during several hours and darkness during the rest of the 24 hours of the day could be suitable. For example, intense lighting during twelve to fourteen hours (or even sixteen hours), followed by twelve to ten hours (or even eight hours) in total or relative darkness will be suitable.

In another embodiment, the first lighting cycle time period T2 is more or less than 24 hours.

The Photosynthetically Active Radiation can for example range up to 500 μmol per square meter per second. Such an intense light promotes mating from the first hours after the filling of the mating cage.

At any time during this step, one or more oviposition devices 2 are attached to the mating cage 1, preferably on its outer side. For example, one removable oviposition device 2 can be attached to the mating cage 1. The attachment of the oviposition devices 2 to the mating cage 1 can also be carried out before the filling of the mating cage 1.

One exemplary embodiment of an oviposition device 2 is represented on FIG. 2. Females seek for shady and dry places to oviposit. As a consequence, the oviposition devices 2 are preferably made of a suitable material and include crevices. The oviposition devices 2 can for example be made of plastic or wood.

For example, an oviposition device 2 can be in the form of a plastic or wooden parallelepiped much less thick than long and wide, having a width and a length inferior to those of the side of the mating cage to which it will be attached. Grooves or trenches 21, having a depth of the order of a few millimeters, machined on the side facing the mating cage 1 will provide ideal conditions (shadow, dryness, etc.) for oviposition. Note that for a better understanding of FIG. 2, the scale is not correct for the dimensions of the trenches.

The trenches can be designed so that eggs can be collected in an automated way.

The face 22 opposite the trenches is turned toward the outside of the mating cage, so that the females in the mating cage 1 can oviposit in the trenches through one or more wall of the mating cage 1.

Chemical or biological substances (pheromones, egg debris, volatile compounds typical of the food required for egg, etc.) can be added to attract gravid females to the oviposition devices 2.

These substances can be placed in specific containers.

These substances can also directly impregnate the surface of the oviposition devices 2.

The oviposition devices 2 can be collected manually or in an automated way.

Therefore, for example, the oviposition devices 2 can slide between two horizontal rails attached to the outer side of the mating cage 1, or the removable attachment can be a mechanical fixing means, like a bayonet attachment of the oviposition devices 2 to the mating cage 1.

In the case the oviposition devices 2 are on the outer face of the mating cage 1, unlike usual practice, no aperture needs to be opened when these devices are collected. This makes it possibly to achieve no adult loss.

However, there is in general no need to collect the oviposition devices 2 during step 2, since few ovipositing occur at this step. This embodiment can be of greater interest at step 3, 4 or 4′.

For the same reasons, only few oviposition devices 2 per mating chamber 1 are required at this step.

The mating cage 1 then enters the third step. If this step takes place in another location, the cage can be automatically moved from the second to the third location.

The third step comprises a second lighting cycle, in order to give the females a second chance of mating, and that also promotes oocytes maturation.

The oviposition devices 2 can be collected during or at the end of step 3. When oviposition devices 2 are collected, new oviposition devices 2 are provided. Additional oviposition devices 2 can also be provided during step 3.

In a particular embodiment, the duration T2b of step 3 is the same as T2.

In general, bright lighting during several hours and darkness during the rest of the 24 hours of the day could be suitable. For example, intense lighting during twelve to fourteen hours (or even sixteen hours), followed by twelve to ten hours (or even eight hours) in total or relative darkness will be suitable.

The mating cage then enters the fourth step. If this step takes place in another location, the cage is automatically moved from the third to the fourth location. During the fourth step, the mating cage undergoes a third lighting cycle (time period T6) that promotes oocytes maturation in the gravid females and oviposition.

Preferably, the light intensity is reduced compared to step 2 and/or step 3, since females require (and search for) shadow places for ovipositing. This disposition is also associated with energy saving.

In an exemplary embodiment, the time period T6 is a 24-hours day

In general, intense lighting during several hours and darkness during the rest of the 24 hours of the day could be suitable. For example, intense lighting during twelve to fourteen hours (or even sixteen hours), followed by twelve to ten hours (or even eight hours) in total or relative darkness will be suitable.

New oviposition devices 2 can be attached to the mating cage at the beginning of step 4. More oviposition devices 2 than in step 2 and/or step 3 can be provided, in so far as step 4 corresponds to the day of peak egg production.

At least some of the oviposition devices 2 can be manually or automatically collected at least at the end of step 4. They can be collected at shorter time intervals, for example if they are satisfactorily filled. For example, the intensity of a light beam having the same direction as one or more trenches before entering and after crossing the one or more trenches can be compared in order to assess the filling of the trenches.

Once the eggs are collected, they can immediately or later enter an egg maturation cycle that will not be described here.

The mating cage 1 can then optionally enter step 4′, which can be similar to step 4. If this step takes place in another location, the cage can be automatically moved from the fourth to the next location. During step 4′, the mating cage undergoes a fourth lighting cycle that promotes oviposition.

In a particular embodiment, the light is less intense than in steps 2 and 3.

In an exemplary embodiment, the duration (or time period) T6b of step 4′ is 24 hours.

In general, moderate lighting during several hours and darkness during the rest of the 24 hours of the day could be suitable. For example, intense lighting during twelve to fourteen hours (or even sixteen hours), followed by twelve to ten hours (or even eight hours) in total or relative darkness will be suitable.

At this stage, oviposition devices 2 are attached to the mating cage 1, on its outer side. For example two or three removable oviposition devices 2 can be attached to the mating cage 1.

The oviposition devices 2 can be manually or automatically collected at least at the end of step 4′. They can be collected at shorter time intervals, for example if they are satisfactorily filled.

Once the eggs are collected, they can enter an egg maturation cycle that will not be described here.

The mating cage 1 can then enter the fifth step. If this step takes place in another location, the cage is automatically moved from the current to the next location. During the fifth step, ovipositing is stopped. This means that the adults no longer have the opportunity to oviposit and no ovipositing device is collected after the beginning of step 5.

In one embodiment, the adults still alive at the beginning of step 5 are killed before their natural death.

For example, step 5 takes place at the end of day 4 after the beginning of step 1. This implies, in the case of Black Soldier Flies, that most of the adults are killed before their natural death (which occurs in general seven to ten days after emergence). In other words, step 5 takes place after a duration less than the adult life expectancy after the beginning of step 1.

The adult life expectancy must be understood as the average duration between emergence and natural death of an insect of a given species.

At the beginning of step 5, some of the adults are already dead, in particular some of the females who have already oviposited, but some of them are still alive.

One aspect of the invention is to consider that, in order to optimize the egg yield, one has to shorten the time spent by the adults in the mating cage 1, on the contrary to usual practice, which consists in waiting for the death of all the adults in a cage.

One will also remember that a female mates at most once. All the eggs that are collected correspond to a mating during days 1, 2 or 3 after the beginning of step 1 if step 5 takes place at the end of day 4.

In a particular embodiment, a calibration is performed so as to decide on the day at which step 5 takes place.

For example, the calibration may comprise, on the same principle as described in the experimental section:
i) filling at least one mating cage 1 with a population of newly emerged insects;
ii) providing conditions suitable for promoting mating and/or oocytes maturation and/or ovipositing within said at least one mating cage 1 for each day of during at least D days (D being a positive integer), for example during three days, or at least four days, or at least five days, or at least six days, or at least seven days, or at least eight days, or at least nine days, or at least ten days (the number of days can of course be adapted depending of the type of insects and in particular depending on their life expectancy. Seven or eight days could be a convenient choice in the case of Hermetica Illucens);
ii) providing at least one oviposition device 2 at least at the beginning of each of the at least D days for each of the at least one mating cage 1;
iv) collecting at least one oviposition device 2 at least at the end of each of the at least D days for each of the at least one mating cage (1);
v) measuring the weights of the eggs collected in each of the oviposition device (2) and determining the duration between the end of a) and the beginning of c) based on the weights measured.

For example, N mating cages 1 undergo the steps i) to v) of the calibration step, N being more than 10, more than 100, more than 500. The eggs are collected and weighed every day during D days after the filling of the mating cage for each mating cage 1. The total weight of eggs collected at day k (k being comprised between 1 and D) for the set of N mating cages 1 is then calculated for each value of k (k being comprised between 1 and D).

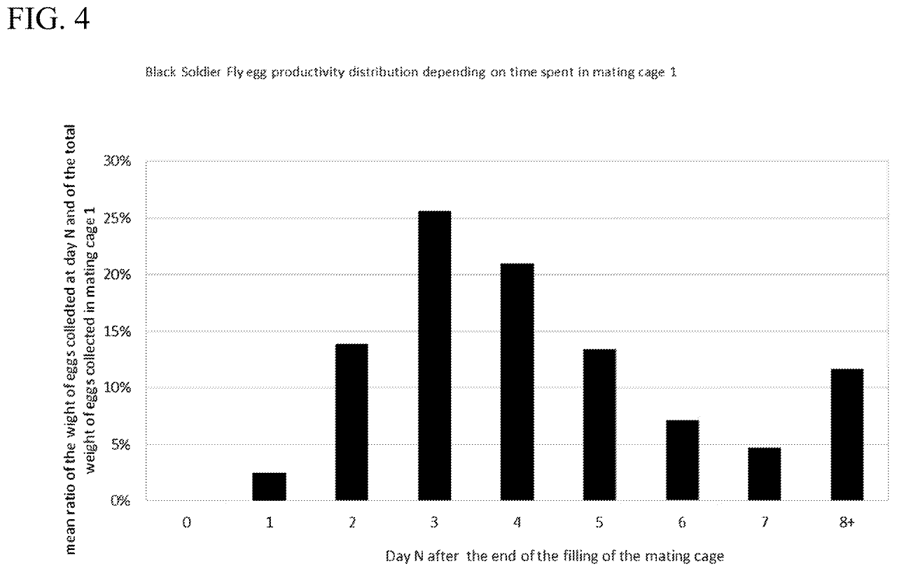

The experimental section and FIG. 4 show clearly that there is no interest in awaiting the death of the adults in the mating cage 1. Table I shows that in the case of the example, less than 12% of the eggs are laid after day 8 and 64% of the eggs are laid before day 4, the maximum oviposition rate being obtained on day 3 and the ovipositing rate continuously decreasing after day 3.

It appears clearly that a higher egg yield can be achieved by stopping ovipositing after day 3 and before day 8 and filling a new mating cage 1 or starting a new cycle rather than awaiting the death of all the adults before filling a new mating cage 1 or starting a new cycle.

The duration between the end of a) and the beginning of c) can thus be determined based for example on the curve of the total weight of eggs collected at day k after the filling of a mating cage 1 as a function of k.

By way of an example, the duration between the end of a) and the beginning of c) can be more than kmax days, kmax being the number of the day corresponding to the maximum of the total weight collected per day, and less than the life expectancy of the adults.

A further advantage of the calibration step is that this calibration step allows a better egg yield on the long term (that is two say if the process is reiterated cyclically) for at least two reasons:
firstly, it is likely that adults corresponding to eggs collected early will lay eggs earlier than adults corresponding to eggs collected lately, both for genetic selection and domestication reasons;
secondly, eggs laid early are more likely to hatch than eggs laid lately.

The duration between the end of a) and the beginning of c) can thus be determined based for example on the curve of the total weight of eggs collected at day k after the filling of a mating cage 1 as a function of k.

By way of an example, the duration between the end of a) and the beginning of c) can be for example more than kmax days but less than (kmax+1) days, less than (kmax+2) days, less than (kmax+3) days, less than (kmax+4) days, less than 2 times kmax.

Most of insects have a nycthemeral rhythm. Therefore, the rhythm of the calibration step is based on a 24-hours day rhythm. However, the skilled man could easily adapt this step to a complete artificial rhythm with an artificial day lasting more or less than 24 hours.

In a particular embodiment, the conditions during at least one of the at least D days of the calibration step are the same conditions as at least one of step 2, step 3, step 4 or step 4′ of the process.

Liquid or gaseous fluid, for example water, can be projected in the mating cage by means of nozzles, so as to drown the insects.

Water vapor is preferred since it makes this step faster and allows simultaneous disinfection of the cage. In this case, the mating cage can be made of a heat resistant mesh such as stainless steel mesh.

In another embodiment, at step 5, the mating cage 1 can be connected to another cage or box and the adults directed to this cage by means of intense lighting in this other cage or box, while the first mating cage 1 is kept in the dark. Once the first mating cage 1 is empty, that is to say contains no living adult, it is disconnected from the other cage or box.

In general, any means which makes it possible to significantly reduce the mating rate and the oviposition rate compared to the rates observed on average under optimal conditions on the date considered after emergence is suitable for the achievement of step 5.

The decision to start step 5 can be made on several basis:
either counting the dead adults. When the percentage of dead adults meets a first threshold, step 5 begins;
or counting the females having oviposited. When the percentage of females having oviposited meets a second threshold, step 5 begins;
or counting the number of eggs collected, or weighing the eggs collected. When the number or the weight of eggs collected meets a third threshold, step 5 begins.

Step 5 doesn't necessarily start as soon as one of the criteria is met. It can start as soon as possible, depending for example on the position of other mating cages 1 in the system.

Maintaining darkness in the mating cage 1, decreasing the temperature, the lack of oviposition devices, etc., can thus be implemented in particular embodiments.

The duration (T3) of step 5 can be less than the durations of any of step 2, 3 4 or 4′. For example, T3 is less than one hour.

The fifth step guarantees that the collected eggs have been oviposited after a mating occurring during the first two days of the female lives. This step provides as a consequence an enhanced control of the quality and characteristics of the eggs collected, and thus a better synchronization of the insects entering the larval stage and, later, a new mating cycle.

In the hypothesis where the mating cycle is coupled with an egg maturation cycle and a larval cycle, after a few cycles corresponding to a transient state, one may consider that synchronization is optimal and the three cycles combined have steady-state production.

As a result, organoleptic qualities or other characteristics of the products of the insects rearing can be relatively stable from batch to batch.

Since not all the adults die before day 5, the precocious termination of the adult phase (that is to say the phase during which the eggs resulting from mating and ovipositing are collected) also limits ovipositing on dead adults, which would result in a lower global egg yield.

In this document, the egg yield is the mass of eggs collected per cubic meter of mating chamber per time unit.

The mating cage 1 can then enter the sixth step. If this step takes place in another location, the cage is automatically moved from the current to the next location.

At this step, the mating cage 1 is cleaned, optionally sterilized, and dried, so as to be reusable for a new step 1.

In case water vapor is projected at step 5, this enables step 6 to take place at the same location and at the same time as step 5. In this case, cleaning the mating cage 1 means also sterilizing this mating cage 1.

In case the sliding door 12 is on the underside of the mating cage 1, it can be reopened at this step to remove debris.

One or more additional steps can be included in the mating cycle.

In another example, step 4 may be repeated one or several times, as a function of the adult life time of the insect bred and the duration of the optimal period for mating at the beginning of the adult stage.

The main limitation is that the adults must be killed before their natural deaths to prevent late mating, late ovipositing, corresponding to poorer egg quality.

In another embodiment, one or more storage steps can be included in the mating cycle.

After the last step, the mating cage 1 can start a new cycle, beginning at step 1. If the mating cage 1 was displaced from step to step, there is possibly no need to remove the mating cage 1 from the conveyor belt (or rail, etc.) between two successive cycles.

For Black Soldier Flies, it has been observed that performing step 5 four days after filling of the mating cage results in 100% fertilized eggs entering the larval stage.

With a cycle as described on FIG. 3, yields of 10 g eggs per cubic meter of mating cage per day or more have been observed.

The temperature and/or relative humidity can be adjusted at each step. In one embodiment, in the case of Black Soldier Flies, the temperature for all the steps between step 1 and step 5 is around 30° C., preferably in the range [25° ° C., 35° C.], and the relative humidity is around 80% for all these steps.

The lighting conditions can be adapted at each step. For example, the spectrum of the light for any of the steps 2 to 4′ may include one or more monochromatic radiations selected from the UV spectrum and/or the visible spectrum. The Photosynthetically Active Radiation can be chosen in the range 80 μmol/m²/s to 500 μmol/m²/s.

Feeding or watering can be provided at one or more steps.

The process which has just been described can be performed in an automated installation.

The automated installation may comprise a plurality of stations each adapted to perform a specific step. According to one example, the stations are provided along a predefined path. The path is cyclic. The automated installation comprises one or more mating cages 1, which undergo the above process cyclically.

A controller system is provided to control the operation of the automated installation. The controller system is adapted to control the cyclic displacement of the mating cages 1 along the plurality of stations. For example, the automated installation comprises a conveyor system to guide the movement of the mating cages along the path. In case of a plurality of mating cages 1, these may be handled synchronously, in a time-shifted manner, so that, at a given time, mating cages are located in various stations, and undergo different steps of the above process.

On or more of the stations may be closed, or partially closed, in order to control the ambient conditions (in particular, temperature, humidity, lighting) in the station when necessary.

“Closed” here means that the closure enables the conditions to be maintained in the station, while being different outside the station, while still not preventing the mating cage 1 from entering or exiting the station.

At each station, a mating cage 1 may be still. Then, the controller system controls the shifting of the mating cage 1 to a next station based on a predefined criterion, such as a predefined schedule and/or a sensed characteristic meeting a predefined condition. In addition, or alternatively, the controller system may control a continuous movement of the mating cage 1 along the path in one or more of the stations.

To allow the controller system to track the mating cages 1, in a particular embodiment, a tracking device can be provided for each mating cage 1. For example, the tracking device is a RFID chip or a barcode.

This tracking device is of particular interest for the filling of a mating cage 1, especially when it is filled with adults from different emergence chambers. For example, it helps monitor the successive connections between one or more mating cage 1 and one or more emergence chambers. As a consequence, it enables an automated population selection for any mating cage 1.

At each station, the controller system may control the operation of the station. This may involve triggering any action based on a predefined criterion, such as a predefined schedule and/or a sensed characteristic meeting a predefined condition. The controller system may therefore be programmed to cause a movement (opening or closing a door, attaching or removing a part) and/or modifying an operational condition (switching lights on or off, modifying ambient temperature, etc.).

A given mating cage 1 being ready for entering a new cycle after step 6 (or optionally step 6b), an automated installation for the imaginal stage cycle according to the invention can comprise several mating cages 1, one or more mating cages 1 being at one or more station at the same time. A given mating cage 1 periodically runs the imaginal stage cycle over a period called Tcycle.

The controller system may further control other cycles of the installation than the imaginal stage cycle of the mating cages 1, in synchronism with this imaginal stage. One particular example is a cycle of emergence cages, which can be controlled by the controller system and synchronized with the cycle of the mating cage 1.

Another example is a cycle of oviposition devices 2, which can be handled in synchronicity with the mating cages 1 to which they are to be assembled to and from which they are to be disassembled, and automatically treated for harvesting laid eggs.

In one embodiment, a tracking device can be provided for each oviposited device 2. For example, the tracking device is an RFID chip or a barcode.

Although particular embodiments of the invention have been disclosed herein in detail, this has been done by way of example and for the purposes of illustration only. The aforementioned embodiments are not intended to be limiting with respect to the scope of the invention. It is contemplated by the inventors that various substitutions, alterations, and modifications may be made to the invention without departing from the spirit and scope of the invention.

### Experimental Section

Each day during three months, 8 mating cages 1 were each filled with 10 000 adults per cubic meter.

Each mating cage 1 was submitted right after the filling to the same series of operational conditions during ten days. Every day during these ten days, oviposition devices 2 were provided and collected in the same way for each mating cage 1.

Every day during these ten days, for each mating cage 1, the laid eggs were collected in the oviposition devices 2 and weighed.

One can see on table 1 for every day after the end of the filling of the mating cage 1 the mean ratio of the weight of eggs laid at day N and the total weight of eggs laid in the mating cage 1 between the end of the filling of the mating cage and the death of all the adults in the mating cage 1, the mean ratio being calculated for the 8*3*30=720 mating cages 1, which represents more than 5 million adults.

| | Daily egg |
|---|---|
| Mating space | productivity |
| age in days | distribution |
| | 0% |
| 1 | 3% |
| 2 | 14% |
| 3 | 26% |
| 4 | 21% |
| 5 | 13% |
| 6 | 7% |
| 7 | 5% |
| 8+ | 12% |
| | Total = 100% |

### LIST OF THE REFERENCE SIGNS

1: mating cage
11: mesh
12: door
13: hook
2: ovipositing device
21: trench for ovipositing
22: face of the ovipositing device opposite the trenches
3: exemplary mating cycle

1. An automated process for breeding insects at their imaginal stage in at least one mating cage, comprising:
1) providing at least one emergence chamber comprising a population of newly emerged insects;
a) automatically successively:
connecting at least one emergence chamber with at least one mating cage,
filling the at least one mating cage with at least part of the population of newly emerged insects during a first time interval T1, and
disconnecting the at least one mating cage and the at least one emergence chamber:
b) providing conditions within at least one of the at least one mating cage suitable for promoting ovipositing during a second time interval T2;
c) stopping ovipositing during a third time interval T3.

2. The process according to claim 1 further comprising performing a calibration before 1), the calibration comprising:
i) filling at least one mating cage with a population of newly emerged insects;
ii) providing conditions suitable for promoting mating and/or oocytes maturation and/or ovipositing within said at least one mating cage during each day of at least three days;
ii) providing at least one oviposition device at least at the beginning of each of the at least three days for each of the at least one mating cage;
iv) collecting at least one oviposition device at least at the end of each of the at least three days for each of the at least one mating cage;
v) measuring the weights of the eggs collected in each of the oviposition device.
vi) determining a duration between the end of a) and the beginning of c) based on the weights measured at v);
a), b) and c) being subsequently performed in such a way as to respect said duration.

3. The process according to claim 1 in which during b), one or more oviposition devices are provided and/or collected without opening the at least one mating cage.

4. The process according to claim 1 further comprising after c):
d) cleaning and emptying at least one mating cage during a fourth time interval T4 so that after d), the at least one mating cage is suitable for a new a);
and optionally:
e) storing the at least one mating cage during a fifth time interval T5.

5. The process according to claim 1 further comprising:
f) providing conditions within the at least one mating cage suitable for promoting mating and/or oocytes maturation and/or ovipositing during a sixth time interval T6 between said b) and c), and in particular wherein said conditions differ from the conditions of b).

6. The process according to 1 in which the duration between the beginning of a) and the beginning of c) is shorter than the life expectancy of the adult insects after emergence.

7. The process according to 1 in which c) starts once at least one of the following criteria is met: the percentage of dead adults meets a first threshold, the percentage of females having oviposited meets a second threshold, the egg yield meets a third threshold.

8. The process according to 1 characterized in that at least two of said time intervals are equal.

9. The process according to 1 characterized in that at least two of 1), a), b), c) and, optionally, d), e), f) take place at at least two different places, and further comprising moving the at least one mating cage from one of said places to another of said places of the at least two different places between two of 1), a), b), c) and, optionally d), e), f).

10. The process according to claim 1 characterized in that at least one said condition of b) or optionally f) is chosen among an ambient temperature, a relative humidity, lighting, feeding and watering.

11. The process according to claim 1 characterized in that the population of newly emerged insects filling the at least one mating cage is selected according to at least one criterion among a sex ratio, a phenotype, a genotype, a size of said insects at the larval stage at a given date, a sexual maturity index and an age.

12. An automated system for breeding insects at their imaginal stage, comprising:
at least one mating cage;
a filling system configured to connect at least one mating cage with at least one emergence chamber comprising a population of newly emerged insects, to fill the at least one mating cage with at least part of the population of newly emerged insects comprised in the at least one emergence chamber during a first time interval T1 and to disconnect the at least one mating cage and the at least one emergence chamber;
a first provision system to provide conditions within the at least one mating cage suitable for promoting ovipositing during a second time interval T2;
a terminating system to stop ovipositing during a third time interval T3.

13. The automated system according to claim 12 further comprising a second provision system to provide conditions within said at least one mating cage suitable for promoting mating and/or oocytes maturation and/or ovipositing.

14. The automated system according to claim 12, wherein
a) said at least one mating cage:
comprises at least one door comprising an automated device for its closing and opening,
is provided with at least one removable ovipositing device, and
has at least two discrete positions, and,
b) said automated system further comprises a moving device configured for moving said at least one mating cage from at least one of said at least two discrete positions to at least another of said at least two discrete positions, each discrete position being equipped to automatically perform at least one of:
a) connecting said at least one mating cage with at least one emergence chamber, filling said at least one mating cage with at least part of the population of newly emerged insects during a first time interval T1, and disconnecting said at least one mating cage from the at least one emergence chamber;
b) providing conditions within said at least one mating cage suitable for promoting ovipositing during a second time interval T2;
c) stopping ovipositing during a third time interval T3;
and optionally
f) providing conditions within at least one mating cage suitable for promoting mating and/or oocytes maturation and/or ovipositing.

15. The automated system according to claim 12 in which each of said at least one mating cage is provided with an automated device for counting the population of newly emerged insects and in which said automated device for closing and opening said door of the respective mating cage receives a signal from said automated device for counting the population of newly emerged insects that triggers the closing and/or opening of the door of the respective mating cage.

16. The automated system according to claim 12 further comprising a controller and wherein a tracking device is provided for each of said at least one mating cage, the controller receiving tracking data from at least one tracking device of a mating cage and controlling the moving of the mating cage based on the tracking data.

17. The automated system according to claim 12 characterized in that at least one removable ovipositing device is provided for each of the at least one mating cage and that said at least one removable ovipositing device is fixed on the outer side of the respective mating cage and is provided with a tracking device, the system further comprising a controller receiving data from at least one tracking device of an ovipositing device and controlling the removal and the fixing of the ovipositing device based on the tracking data received from the tracking device of the ovipositing device.

18. A system comprising an automated system for breeding insects at their imaginal stage according to claims 12-17 coupled with a system for breeding insects at the egg stage and/or at the pupation stage.
